(19) United States
(12) Patent Application Publication
(10) Pub. No.: US 2005/0277838 A1
(43) Pub. Date: Dec. 15, 2005

(54) Noninvasive biometric measuring device

(75) Inventors: Yutaka Ikeda, Kakogawa (JP); Toshiyuki Ozawa, Miki -shi (JP); Takeo Saitou, Kobe-shi (JP)

Correspondence Address: BIRCH STEWART KOLASCH & BIRCH, PO BOX 747, FALLS CHURCH, VA, 22040-0747, US

(73) Assignee: SYSMEX CORPORATION

(21) Appl. No.: 11/149,274

(22) Filed: Jun. 10, 2005

(30) Foreign Application Priority Data
Jun. 15, 2004 (JP) 2004-177272

(51) Int. Cl. A61B 5/02

(52) U.S. Cl. 600/500; 600/503

(57) ABSTRACT

Noninvasive biometric body measuring devicees are described, a representative one of which includes: an obtaining part obtaining biometric information from the measured region of a living body; a pressing part for pressing the living body close to the measured region; a pressure sensor detecting a press force of the pressing part; a control part controlling the press force of the pressing part based on a detected value of the pressure sensor; and an analyzing part analyzing the biometric information obtained by the obtaining part in such a state that the living body is pressed by the pressing part.

### FIELD OF THE INVENTION

The present invention relates to a noninvasive biometric measuring device. More specifically, the present invention relates to an device analyzing information optically obtained from part of a living body to measure blood information, e.g., hemoglobin concentration.

### BACKGROUND

As the background art of the present invention, there is a known biometric information measuring device which has light-emitting means for applying light to a living body, light receiving means for receiving reflected light from the living body of the light applied by the light-emitting means to produce a biometric information signal according to the amount of the light received, and living body pressing means disposed on a portion closer to the heart of the living body than the light receiving means and adapted to adhere to the living body to press the living body, the pressing means for mitigating fluctuations on the periphery side caused by movement of the living body (for instance, see U.S. Pat. No. 6,529,754).

There is also a known device in which near infrared rays are irradiated from a light source to a part being measured of a living body in such a state that vein bloodstream at the part being measured is blocked by applying a press force of a cuff, the intensities of the transmitted near infrared rays are detected by a photodetector, and the blood sugar level of the living body is determined on the basis of the absorbance of the near infrared rays at the part being measured (for instance, see U.S. Pat. No. 6,149,588).

The noninvasive biometric measuring device is simple since it enables percutaneous blood analysis and permits continuous monitoring. It is desirable to increase the accuracy in analysis to a degree comparable to blood analysis of conventional blood sampling. In the biometric measuring devicees disclosed in U.S. Pat. No. 6,529,754 and U.S. Pat. No. 6,149,588, biometric information is obtained in such a state that a living body is pressed by the living body pressing means or the cuff. A specific configuration for constantly maintaining the press force during measurement is not disclosed.

### SUMMARY

The present invention has been made in view of such circumstances. The aim of the present invention is to provide a noninvasive biometric measuring device properly adjusting a press force applied to a living body, thereby increasing the accuracy in analysis.

The noninvasive biometric measuring device of a first aspect of the present invention includes: an obtaining part for obtaining biometric information from a measured region of a living body; a pressing part for pressing the living body close to the measured region; a pressure sensor for detecting a press force of the pressing part; a control part for controlling the press force of the pressing part based on a detected value of the pressure sensor; and an analyzing part for analyzing the biometric information obtained by the obtaining part in such a state that the living body is pressed by the pressing part.

The noninvasive biometric measuring method of a second aspect of the present invention includes the steps of: pressing a pressed portion closer to a measured region of a living body; detecting a press force applied to the pressed portion; controlling the press force based on a detected value; obtaining biometric information from the measured region of the living body in such a state that the pressed portion is pressed; and analyzing the obtained biometric information.

The living body pressing device of a third aspect of the present invention includes: a pressing part adapted to adhere to a living body to press the living body; a pressure sensor detecting a press force of the pressing part; and a control part controlling the press force of the pressing part based on a detected value of the pressure sensor, wherein the control part calculates a press force applied to the living body based on said detected value to control the press force applied to the living body based on said press force.

### BRIEF DESCRIPTION OF THE DRAWINGS

FIG. 1 is a top view showing the essential part of an embodiment of the present invention;

FIG. 2 is a side view showing the essential part of an embodiment of the present invention;

FIG. 3 is a cross-sectional view taken along the line A-A of FIG. 1;

FIG. 4 is a view taken along the line B-B of FIG. 3;

FIG. 5 is an explanatory view showing arrangement to part of a living body of an embodiment of the present invention;

FIG. 6 is a block diagram showing the configuration of an embodiment of the present invention;

FIG. 7 is a flowchart showing the operation of an embodiment of the present invention;

FIG. 8 is an explanatory view showing image processing of an embodiment of the present invention;

FIG. 9 is an explanatory view showing image processing of an embodiment of the present invention;

FIG. 10 is an explanatory view showing image processing of an embodiment of the present invention;

FIG. 11 is an explanatory view showing image processing of an embodiment of the present invention;

FIG. 12 is a graph showing the level of matching of actually measured values with calculated values;

FIG. 13 is a perspective view showing the configuration of an embodiment of the present invention;

FIG. 14 is an explanatory view showing the essential configuration of an embodiment of the present invention;

FIG. 15 is a flowchart showing the essential operation of an embodiment of the present invention;

FIG. 16 is a flowchart showing the essential operation of an embodiment of the present invention;

FIG. 17 is a flowchart showing the essential operation of an embodiment of the present invention;

FIG. 18 is a flowchart showing the essential operation of an embodiment of the present invention;

FIG. 19 is a flowchart showing the essential operation of an embodiment of the present invention; and

FIG. 20 is a flowchart showing the essential operation of an embodiment of the present invention.

### DETAILED DESCRIPTION OF THE PREFERRED EMBODIMENT

Embodiments of the present invention will be described below in detail based on the drawings. This does not limit the present invention.

FIG. 13 is a perspective view showing a configurational example of a noninvasive biometric measuring device according to this embodiment. It includes a biometric measuring device 5 and a living body pressing device 4. The biometric measuring device 5 has a detecting part 1, an analyzing part 2, a cable 3, an input part 28, and an output part 17. The detecting part 1 fitted on wrist WR of a human (subject) is connected via the cable 3 to the analyzing part 2. The analyzing part 2 is connected to the input part 28. Information from the detecting part 1 is output via the analyzing part 2 to the output part 27. The living body pressing device 4 has a pressing band 101, an air tube 103, and a pressing controller 102. The pressing band 101 is connected via the air tube 103 to the pressing controller 102. The pressing band 101 such as a cuff (pressing band for measuring blood pressure) is wound around and presses the arm portion of the subject, that is, a portion closer to the heart than the wrist WR. As described later, the bloodstream of the blood vessel (vein) of the wrist WR is controlled.

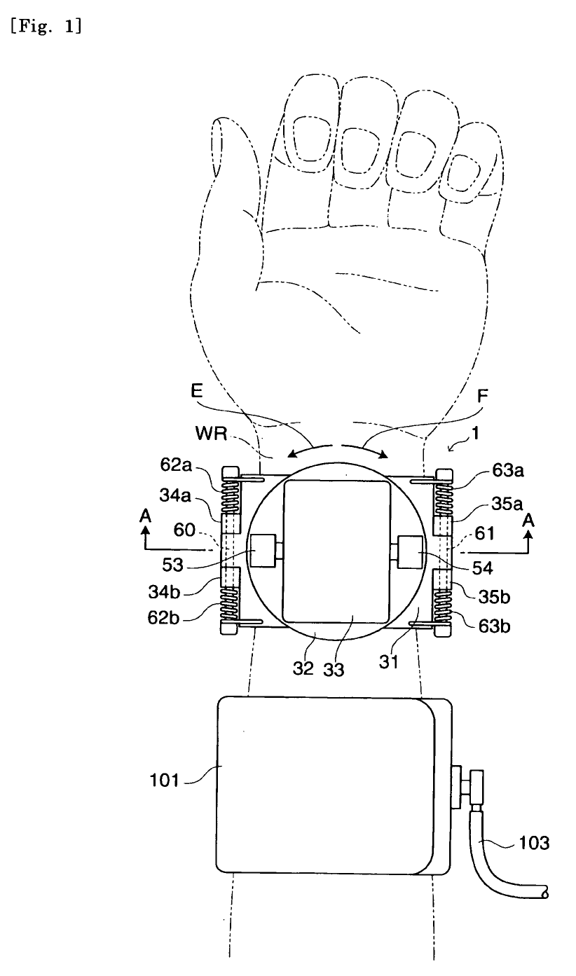

FIG. 1 is a top view of the detecting part 1 fitted on the wrist WR.

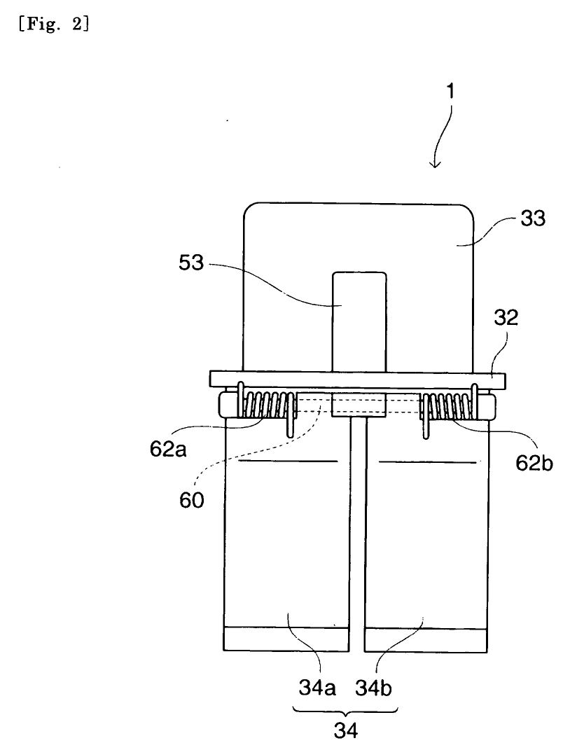

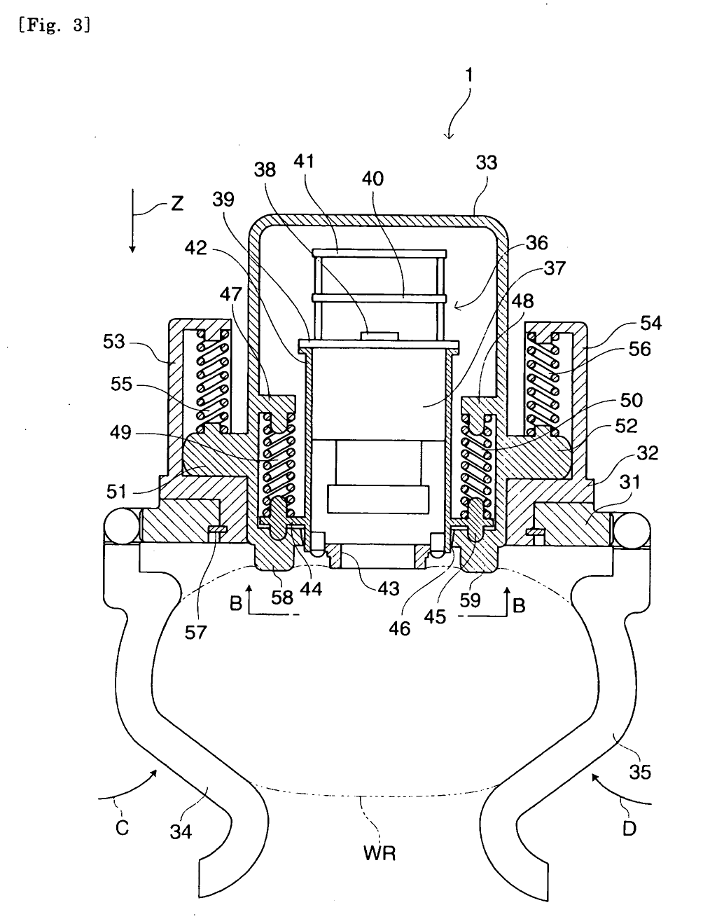

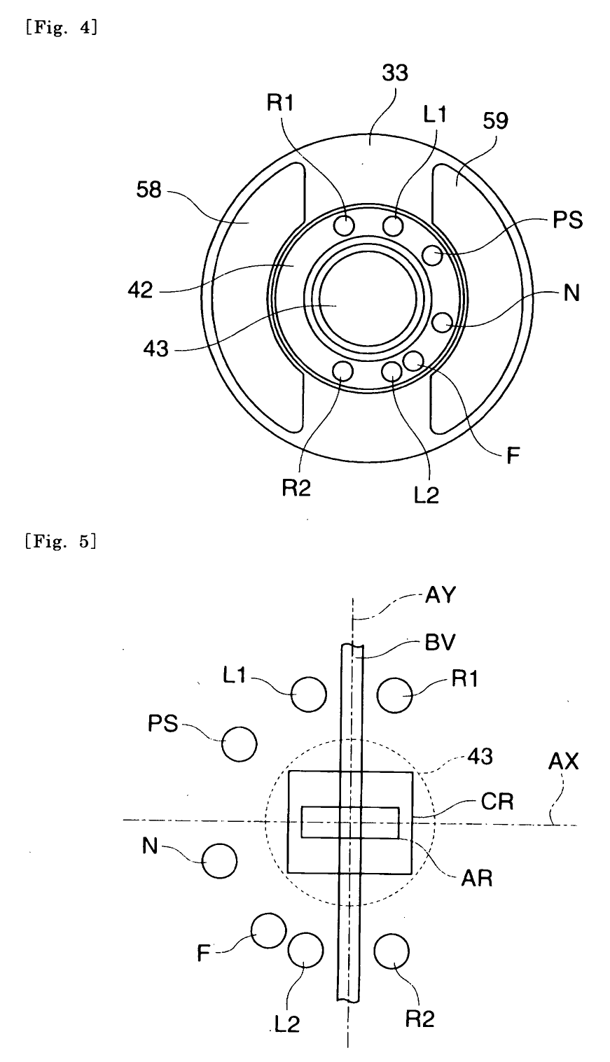

FIG. 2 is a side view of the detecting part 1. FIG. 3 is a cross-sectional view taken along the line A-A of FIG. 1. FIG. 4 is a view taken along the line B-B of FIG. 3.

As shown in these drawings, the detecting part 1 has a support base 31, a rotating-base 32 vertically inserted into the center opening of the support base 31 and rotatably supported in arrow E and F directions (FIG. 1), a housing 33 fitted in the center opening of the rotating base 32, and a pair of holding pieces 34 and 35 for fixing the support base 31 to the wrist WR.

As shown in FIG. 3, the housing 33 has at its bottom an opening 46 and houses an imaging part 36. The imaging part 36 has a cylindrical lens tube 37 incorporating an objective lens, a substrate 39 mounting a CCD image device 38, and substrates 40 and 41 mounting the driving electronic component of the CCD image device 38. The imaging part 36 is inserted into a cylindrical support member 42 to be vertically supported.

The support member 42 has at its bottom a circular opening 43. As shown in FIG. 4, six light-emitting diodes R1, R2, L1, L2, N, and F and one photo sensor PS are arranged around the circular opening 43 on the circumference concentric with the opening 43. The support member 42 has a pair of engagement parts 44 and 45 protruded horizontally from the outer wall surface at the lower end. The engagement parts 44 and 45 are engaged with the circumference of the bottom of the housing 33 so that the opening 43 is protruded downwardly from an opening 46 at the bottom of the housing 33.

The housing 33 has a pair of protrusions 47 and 48 protruded horizontally from the inner wall surface. The protrusion 47 and the engagement part 44, and the protrusion 48 and the engagement part 45 are connected by compression springs 49 and 50. The springs 49 and 50 press the engagement parts 44 and 45 in arrow Z direction, respectively.

The housing 33 has a pair of engagement parts 51 and 52 protruded horizontally from the outer wall surface. The engagement parts 51 and 52 are engaged with the circumference of the opening of the rotating base 32. The rotating base 32 has a pair of spring housing parts 53 and 54 on its top surface. The spring housing parts 53 and 54 house accommodating springs 55 and 56 pressing the engagement parts 51 and 52 in the arrow Z direction.

A ring-like elastic member 57 is fitted at the interface of the inner circumferential surface of the opening of the support base 31 and the rotating base 32. The elastic member 57 functions as a retainer preventing the rotating base 32 from going out of the support base 31 upward and functions as a friction member giving a suitable frictional force between the support base 31 and the rotating base 32 at the rotation of the rotating base 32.

The housing 33 comprises a pair of protrusions 58 and 59 protruded downwardly from the circumference of the opening 46. The protrusions 58 and 59 are contacted with the surface of the wrist WR. The elasticity of the springs 55 and 56 presses the surface of the wrist WR by a suitable pressure. The opening part 43 is also contacted with the surface of the wrist WR. The elasticity of the springs 49 and 50 presses the surface of the wrist WR by a suitable pressure. The protrusions 58 and 59 and the opening 43 can be contacted with the surface of the wrist WR without pressing the blood vessel.

As shown in FIG. 2, the holding piece 34 is divided into segments 34a and 34b and is pivoted on the support base 31 by a through pin 60. The pin 60 is provided with springs 62a and 62b pressing the segments 34a and 34b in arrow C direction respectively (FIG. 3).

The holding piece 35 is also divided into segments 35a and 35b (FIG. 1) and is pivoted on the support base 31 by a through pin 61. The pin 61 is provided with springs 63a and 63b pressing the segments 35a and 35b in arrow D direction respectively (FIG. 3).

The springs 62a, 62b, 63a, and 63b press the segments 34a, 34b, 35a, and 35b by a force securely fixing the support base 31 onto the wrist WR. The holding pieces 34 and 35 are divided into two segments, respectively. Thus, when a protrusion (the bone of the wrist) exists in the fitted portion, the support base 31 can be stably fitted on the wrist WR.

The optical axis of the objective lens incorporated in the lens tube 37, the center axis of the circular opening 43, and the rotating axis of the rotating base 32 are matched with each other.

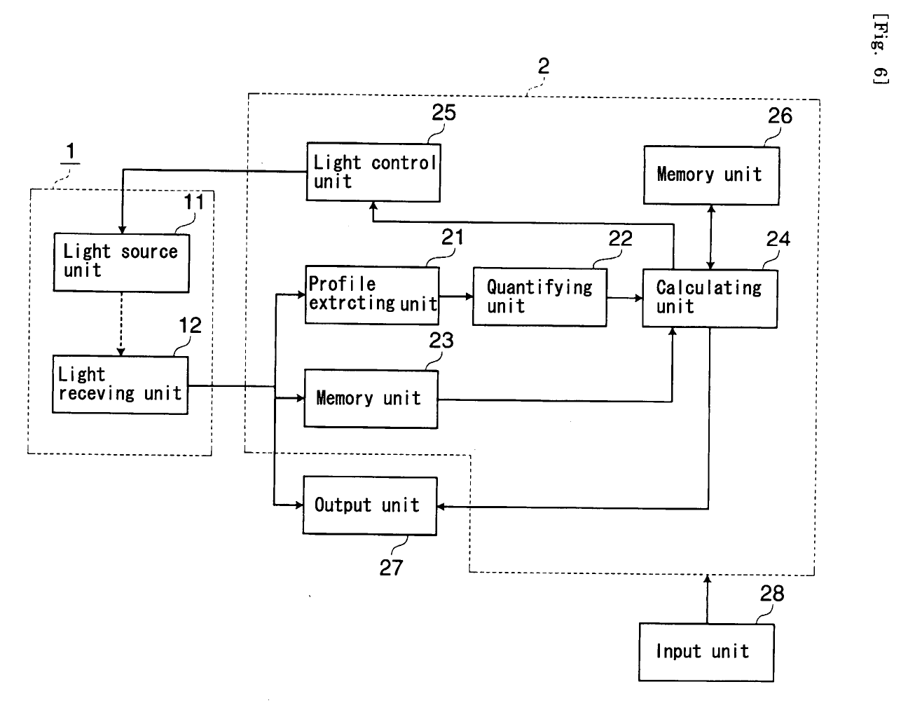

FIG. 6 is a block diagram showing the configuration of the noninvasive biometric measuring device of the present invention.

As shown in the drawing, the biometric measuring device has the detecting part 1, the analyzing part 2, and the input part 28. The detecting part 1 has a light source part 11 and a light receiving part 12. The light source part 11 has the six light-emitting diodes R1, R2, L1, L2, N, and F (FIG. 4). The light receiving part 12 has the CCD image device 38 (FIG. 3) and the photo sensor PS (FIG. 4).

When fitting the detecting part 1 on the wrist WR of a human as shown in FIG. 1, a measured region surrounded by the light-emitting diodes R1, R2, L1, L2, N, and F is formed, as shown in FIG. 5. The light-emitting diodes R1, R2, L1, and L2 are arranged to be symmetrical with respect to a first axis AY and a second axis AX passing through the center of the opening 43 and orthogonal to each other. The light-emitting diodes R1, R2, L1, and L2 illuminate blood vessel BV from both sides. The light-emitting diodes N and F illuminate a region not including the blood vessel BV. The CCD image device 38 images a light image (herein, a reflected light image) of imaged region CR including the illuminated blood vessel BV. The photo sensor PS measures the amount of light inputted from the living body portion not including the blood vessel BV via the light-emitting diodes N and F to the photo sensor. The light-emitting diode N is provided closer to the photo sensor PS than the light-emitting diode F.

A profile extracting part 21 extracts an image density distribution of analyzed region AR (FIG. 5) of an image imaged by the CCD image device 38 of the light receiving part 12 as a brightness profile. A quantifying part 22 quantifies the formative characteristic of the extracted brightness profile. A storage part 23 converts optical information obtained from the light receiving part 12 to digital data to store it.

A calculating part 24 calculates the concentration of the blood constituents based on the quantified characteristic and data of the amount of light. A light control part 25 properly feedback controls the amount of light of the light source part 11 based on information obtained from the light receiving part 12. A storage part 26 stores the result calculated by the calculating part 24. An output part 27 outputs the calculated result and a monitored image. The input part 28 is constituted of such as a keyboard and a mouse and inputs setting of a measuring condition and a calculating condition. The input part 28 comprises a start key for starting measurement.

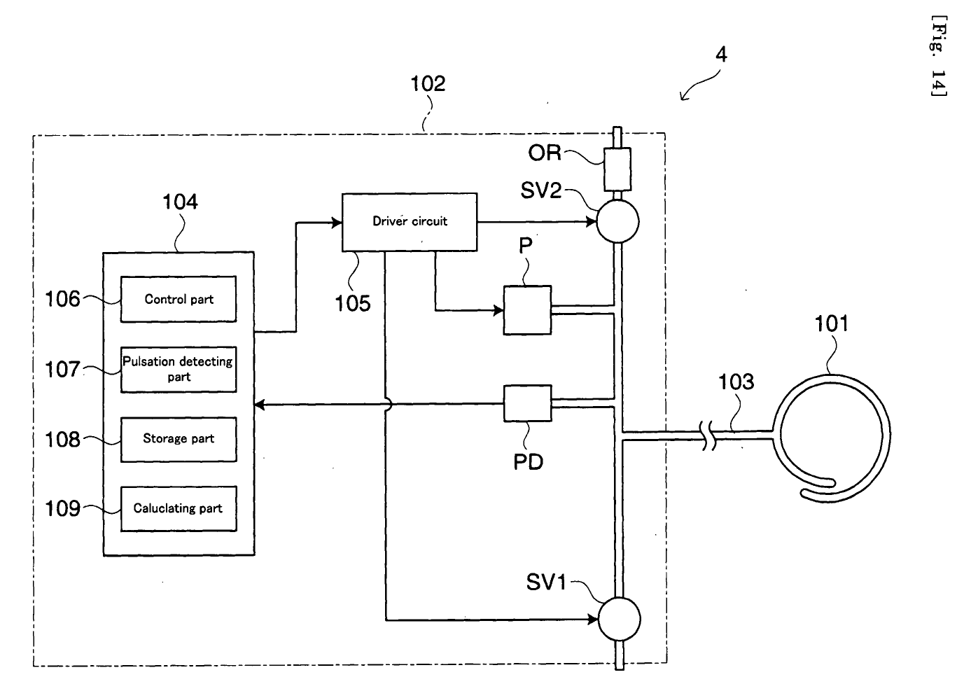

FIG. 14 is an explanatory view of the configuration of a pressure controller controlling a press force of the pressing band 101.

The pressure controller 102 is connected via the air tube 103 to the pressing band 101 to supply or exhaust air to/from the pressing band 101. The pressure controller 102 comprises air pump P, electromagnetic valves SV1 and SV2, pressure sensor PD, orifice OR, a controller 104, and a driver circuit 105. The pressure controller 102 comprises a start key and a stop key, not shown.

The air pump P is connected to the air tube 103 and is also connected to the electromagnetic valves SV1 and SV2 and the pressure sensor PD. The air pump P is driven to supply air to the pressing band 101. The electromagnetic valve SV 1 is opened to release air in the pressing band 101 into the atmosphere. The electromagnetic valve SV2 is opened to gradually release air in the pressing band 101 via the orifice OR into the atmosphere. The controller 104 comprises a control part 106, a pulsation detecting part 107, a storage part 108, and a calculating part 109 and is constituted of a microcomputer. The pulsation detecting part 107 receives an output from the pressure sensor PS to detect a pressure for detecting the presence or absence of pulsation of the detected pressure. The storage part 108 stores processed data. The calculating part 109 calculates processed data. The control part 106 controls the driver circuit 105 to drive the air pump P and the electromagnetic valves SV1 and SV2.

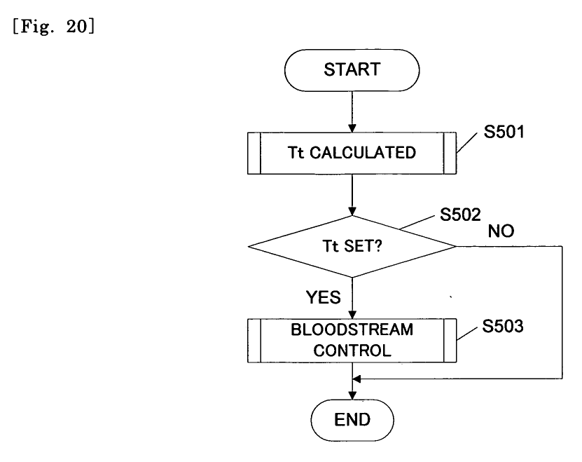

The calculation setting processing of optimum target value Tt of a pressure and the bloodstream control processing by the pressure controller 102 of the pressing band 101 in such configuration will be described using the flowchart shown in FIG. 20.

As shown in FIG. 13, the pressing band 101 is wound around the arm of a subject. In step S501, the controller 104 (see FIG. 14) calculates the optimum target value Tt of a pressure of the pressing band 101. When calculating the optimum target value Tt, the routine is advanced to step S502. In step S502, the controller 104 judges whether the optimum target value Tt is to be set or not. When judging that the optimum target value Tt is to be set, the routine is advanced to step S503. When judging that the optimum target value Tt is not to be set, the processing is ended. In step S503, the controller 104 controls the pressure of the pressing band 101 based on the optimum target value Tt.

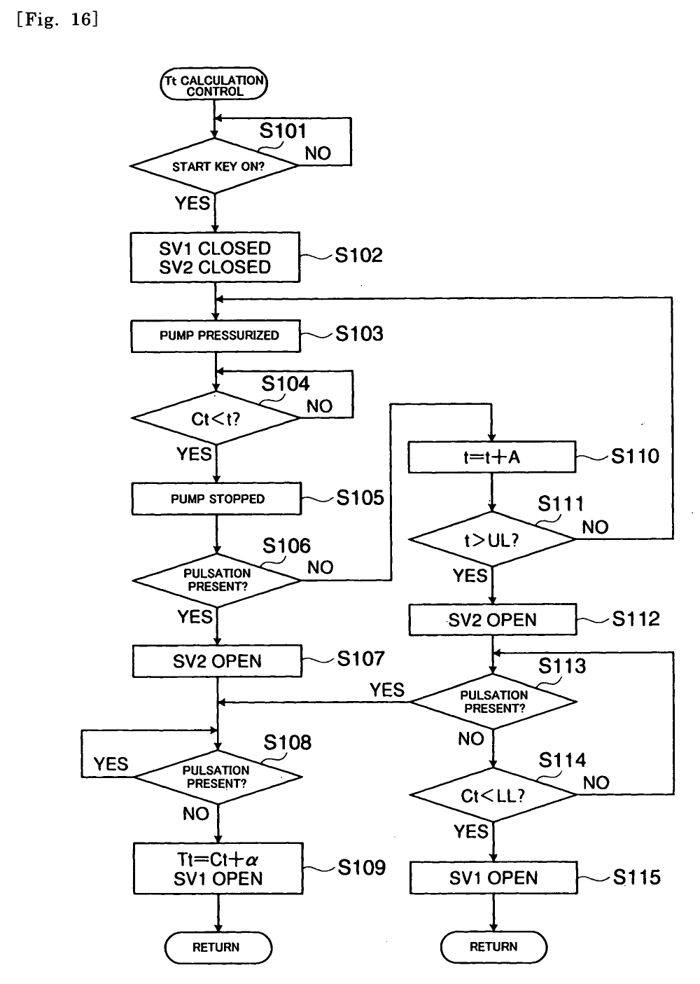

Next, three methods corresponding to the “Tt calculation setting” processing in step S502 (FIG. 20) will be described using the flowcharts of FIGS. 16, 17, and 18.

In other words, when the pressing band 101 is wound around the arm of a subject, any one of the processes shown in FIGS. 16, 17, and 18 is executed.

In the method shown in FIG. 16, the controller 104 (see FIG. 14) judges whether the start key, not shown, of the pressure controller 102 is on or not (step S101). When the start key is on, the routine is advanced to step S102. In step S102, the controller 104 closes the valves SV1 and SV2 via the driver circuit 105. Then, the air pump P is driven (step S103). When pressure Ct of the pressing band 101 is detected by the pressure sensor PD so that Ct≧t (t is a set value) (step S104), the air pump P is stopped (step S105).

The pulsation detecting part 107 judges whether pulsation is present in the output of the pressure sensor PD or not (step S106). When pulsation is present, the valve SV2 is opened (step S107). Air in the pressing band 101 is gradually released into the atmosphere. The pressure Ct when there is no pulsation is detected by the pressure sensor PD and is stored in the storage part 108.

Wherein, the optimum target value Tt is calculated as Tt=Ct+α (α is an experimentally determined constant) by the calculating part 109 and is stored. The valve SV1 is opened to release air in the pressing band 101 into the atmosphere (steps S108 and S109). When no pulsation is present in step S106, set value t is increased to t+A (step S110). When t is not larger than upper limit UL (step S111), the routine is returned to step S103.

When t is larger than the upper limit value UL, the valve SV2 is opened to gradually release air in the pressing band 101 into the atmosphere (step S112). When pulsation is present (step S113), the routine is returned to step S108. When the pressure Ct of the pressing band 101 is smaller than lower limit value LL in the state that no pulsation is present (step S114), the valve SV 1 is opened (step S115). In this case, the optimum target value Tt is not calculated.

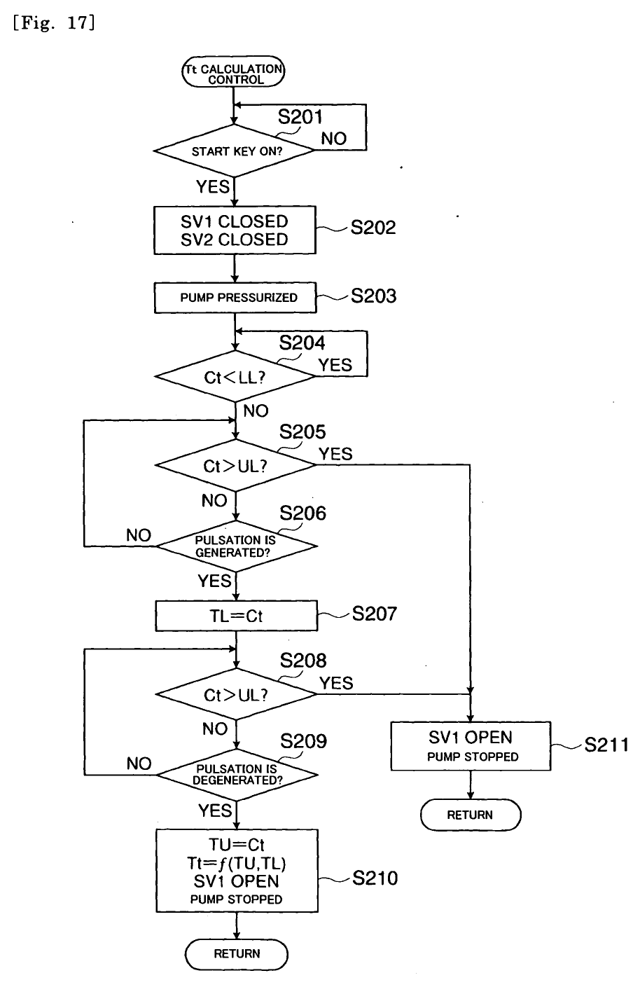

In the method shown in FIG. 17, in step S201, the controller 104 (see FIG. 14) judges that the start key, not shown, of the pressure controller 102 is on. The valves SV1 and SV2 are closed (S202). The air pump P is driven (step S203). The pressure Ct is gradually increased so that Ct is larger than the lower limit value LL (step S204). When pulsation is detected by the time the Ct reaches the upper limit value UL (steps S205 and S206), the pressure Ct when beginning to detect pulsation is stored as minimum proper pressure TL (step S207).

When the pump P is driven so that there is no pulsation by the time the pressure Ct reaches the upper limit value UL (steps S208 and S209), the pressure Ct when pulsation begins to disappear is stored in the storage part 108 as maximum proper pressure TU. The optimum target value Tt is calculated as Tt=f (TU, TL) by the calculating part 109 and is stored. The valve SV1 is opened to stop the air pump P (step S210). In steps S205 and S208, when the pressure Ct is above the upper limit value UL, the valve SV1 is opened to stop the air pump P (step S211). In this case, Tt is not calculated.

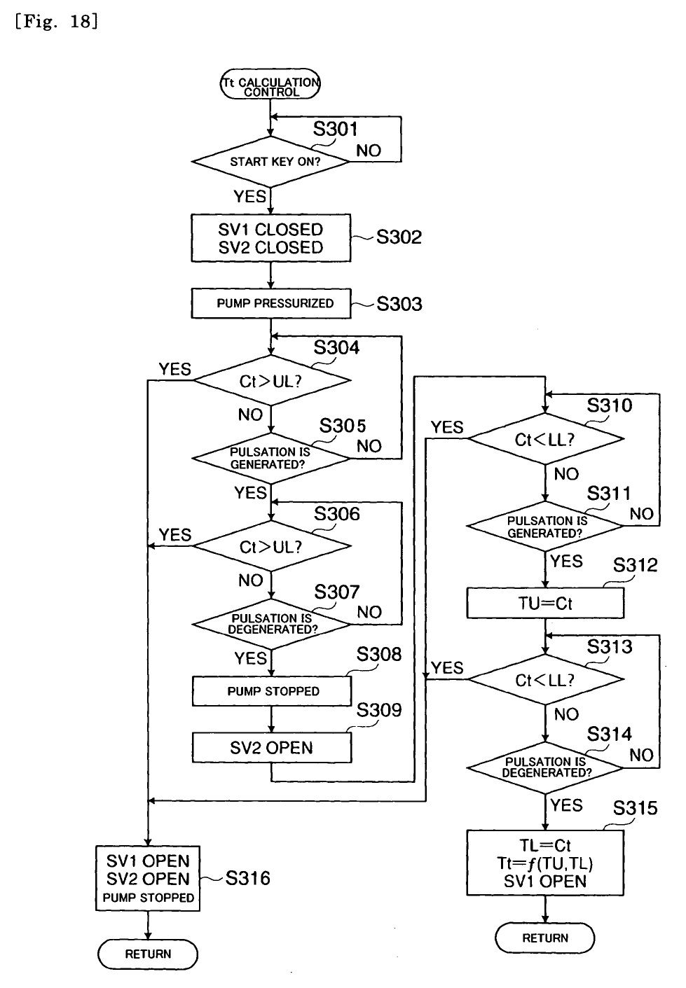

In the method shown in FIG. 18, in step S301, the controller 104 (see FIG. 14) judges that the start key, not shown, of the pressure controller 102 is on. The valves SV1 and SV2 are closed to drive the air pump P (steps S302 and S303). The pressure Ct is gradually increased to detect pulsation by the time Ct reaches the upper limit value UL (steps S304 and S305). When pulsation disappears by the time Ct reaches the upper limit value UL (steps S306 and S307), the pump P is stopped to open the valve SV2 (steps S308 and S309).

The pressure Ct began to decrease gradually. When pulsation appears until Ct reaches the lowest limit value LL, the pressure Ct at the appearance point is stored as the maximum proper pressure TU (steps S310 to S312).

Furthermore, the pressure Ct is lowered so that pulsation disappears by the time Ct reaches the lowest limit value LL (steps S313 and S314). The pressure Ct at the disappearance point is stored as the minimum proper pressure TL. The optimum target value Tt is calculated as Tt=f (TU, TL) and is stored to open the valve SV1 (step S315).

When the pressure Ct is above the upper limit value UL in steps S304 and S306 and when the pressure Ct is below the lower limit value LL in steps S310 and S313, the valves SV1 and SV2 are opened to stop the pump P and the optimum target value is not calculated (step S316).

In FIGS. 17 and 18, as the function Tt=f (TU, TL) calculating the optimum target value Tt, there are:
Tt=(TU+TL)/2; and
Tt=a·TU+b·TL (a and b are constants.)

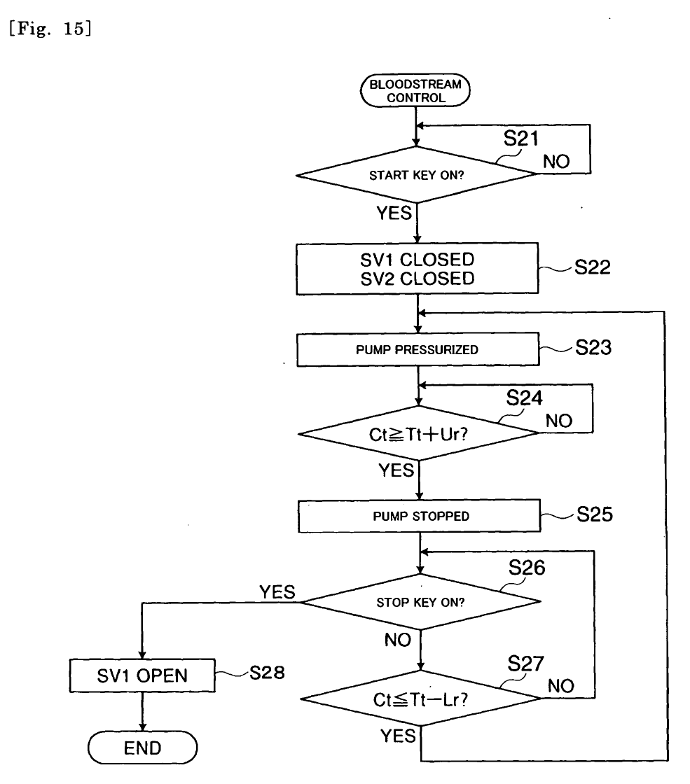

The “Bloodstream control” processing in step S502 (FIG. 20) will be described in detail using the flowchart of FIG. 15. The controller 104 (see FIG. 14) judges whether the start key, not shown, of the pressure controller 102 is on or not (step S21). When the start key is on, the routine is advanced to step S22. In step S22, the controller 104 closes the valves SV1 and SV2 via the driver circuit 105.

The air pump is driven (step S23). The pressure Ct of the pressing band 101 is detected by the pressure sensor PD. When the pressure Ct is above the predetermined optimum target value Tt by upper limit tolerance Ur, the air pump P is stopped so that the pressure controller 102 notifies that measurement can be made (step S25). When the later-described measuring processing is performed to end measurement, the stop key, not shown, of the pressure controller 102 is turned on. The controller 104 (see FIG. 14) judges whether the stop key of the pressure controller 102 is on or not (step S26). When the stop key is on, the routine is advanced to step S28. In step S28, the valve SV1 is opened to release air in the pressing band 101 into the atmosphere. In step S26, when judging that the stop key is not on, the routine is advanced to step S27. In step S27, the pressure Ct of the pressing band 101 is monitored. When the pressure Ct is below the target value Tt by lower limit tolerance Lr, the routine is returned to step S23. In other words, when the pressure Ct is decreased, the air pump P is driven to increase the pressure Ct of the pressing band 101. Thus, in the “bloodstream control” processing, the pressure Ct is maintained in the optimum pressure range of Tt−Lr≦Ct≦Tt+Ur.

The measuring processing of hemoglobin concentration will be described. In the state that the pressure controller 102 presses a wrist to the optimum target value by the pressing band 101 to control the bloodstream of the blood vessel (vein) of the wrist, the detecting part 1 is fitted on the wrist as shown in FIG. 1 to adjust the position of a measured region. In this case, while observing a monitored image output from the output part 27, the housing 33 is rotated in the arrow E or F direction (FIG. 1) to adjust the position of the imaged region CR. As shown in FIG. 5, the targeted blood vessel BV is arranged between the light-emitting diodes R1 and L1 and between the light-emitting diodes R2 and L2.

The analyzing part 2 (see FIG. 13) judges whether the start key, not shown, of the input part 28 is on or not (step S1). When the start key is on, the routine is advanced to step 3. In step 3, the light control part 25 and the light source part 11 illuminate the measured region of part of a living body (here, the wrist of a human) including the blood vessel BV by the light-emitting diodes R1, R2, L1, and L2 (right and left illuminating mode) in the right amount of light to image the imaged region CR (FIG. 5) by the CCD image device 38. Thereby, the image of tissues including the image of the blood vessel (vein) BV in the imaged region CR is obtained.

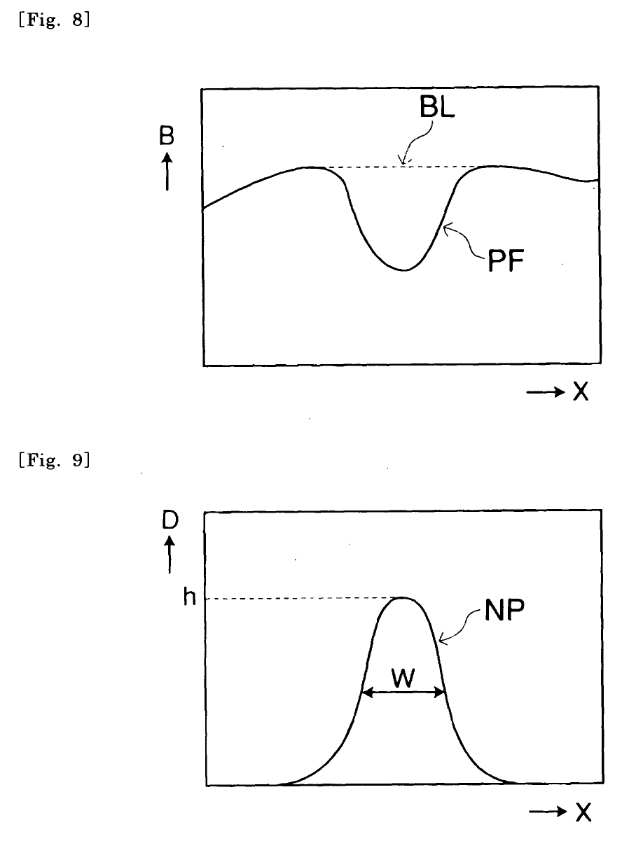

Then, the profile extracting part 21 creates a brightness profile crossing the blood vessel BV in the analyzed region AR (FIG. 5) (a distribution of brightness B to position X), as shown in FIG. 8, to reduce noise components using the fast Fourier transformation method (step S4).

The quantifying part 22 standardizes the brightness profile PF obtained in step S4 by baseline BL (FIG. 8). The baseline BL is determined based on the shape of the brightness profile PF of the absorbed portion of the blood vessel. Thus, concentration profile not dependent on the amount of incident light (a distribution of density D to position X) NP as shown in FIG. 9 can be obtained (step S5).

The calculating part 24 calculates peak height h and half-value width w of the standardized concentration profile NP. In other words, h obtained here expresses the ratio of light intensity absorbed by the blood vessel (blood) of the measured target to light intensity passing through the tissue portion. w expresses a length corresponding to the diameter of the blood vessel (step S6).

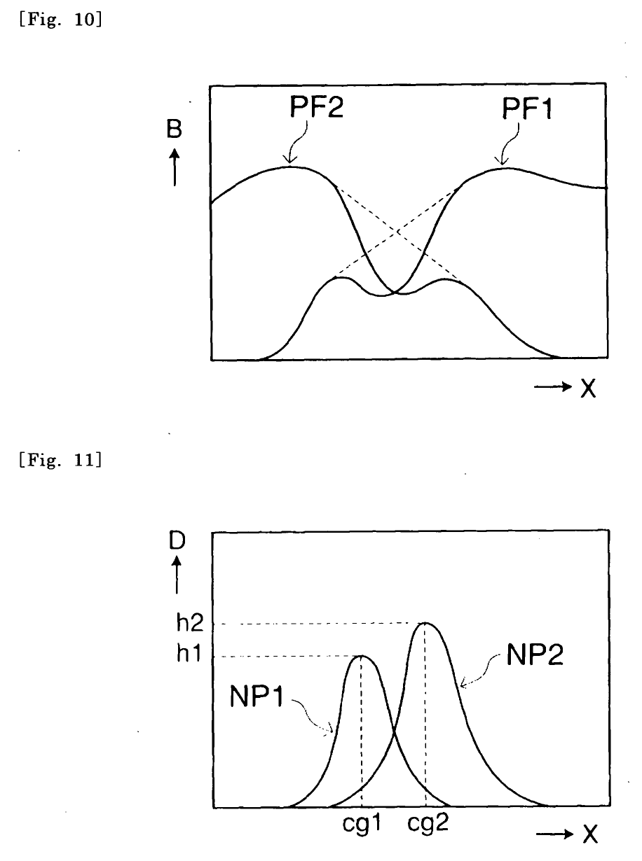

The portion imaged in step S3 is illuminated by the light-emitting diodes R1 and R2 (right illuminating mode) in the proper amount of light and is imaged. Subsequently, it is illuminated by the light-emitting diodes L1 and L2 (left illuminating mode) in the right amount of light and is imaged (step S7). Then, the profile extracting part 21 subjects the respective images obtained in step S7 to the same processing as step S4 to obtain the brightness profiles PF1 and PF2 as shown in FIG. 10 (step S8).

The quantifying part 22 subjects the brightness profiles PF1 and PF2 obtained in step S8 to the same processing as step S5 to obtain the concentration profiles NP1 and NP2 not dependent on the amount of incident light (FIG. 11) (step S9).

The calculating part 24 calculates peak height h1 and center-of-gravity coordinates cg1 from the concentration profile NP 1 obtained by illumination of the light-emitting diodes R1 and R2 and peak height h2 and center-of-gravity coordinates cg2 from the concentration profile NP2 obtained by illumination of the light-emitting diodes L1 and L2 (step S10).

The calculating part 24 calculates blood vessel part scattering amount index S expressed by the following calculating expression using the result obtained in step S10 (step S11).
S=(cg2−cg1)/[(h1+h2)/2] (1)

The light control part 25 and the light source part 11 illuminate the living body portion near the imaged region CR by the light-emitting diode N in the right amount of light. The photo sensor PS measures the amount of light v1 of light incident via the living body portion. The measured result is stored in the storage part 23 (step S12).

The light control part 25 and the light source part 11 illuminate the light-emitting diode F in the same amount of light as that illuminating the light-emitting diode N in step S12. The amount of light v2 of light incident on the photo sensor PS is measured and stored, as in step S12 (step S13).

The calculating part 24 calculates tissue blood amount index D expressed by the following calculation expression using the result obtained in steps S12 and S13 (step S14).
D=log(v1/v2) (2)

The calculating part 24 judges whether the blood vessel part scattering amount index S obtained in step S11 and the tissue blood amount index D obtained in step S14 satisfy the following condition or not (step S15).
a1*Sb≦D≦a2*Sb (3)
(Wherein, a1<a2, a1, a2, and b are experimentally obtained constants.)

When not satisfying the condition of the equation (3), the calculating part 24 judges that the reliability of the measured result is low to perform measurement again or stop measurement. When satisfying the condition of the equation (3), the calculating part 24 judges that the reliability of the measured result is high so that the routine is advanced to the next step S16.

The calculating part 24 decides a correction factor using D and an experimentally obtained correction calibration curve. Assuming that the Berr law is approximately established, the blood concentration in the blood vessel is calculated from h and w. A value obtained by multiplying the blood concentration by the correction factor is calculated as hemoglobin concentration HGB to be stored in the storage part 23 (step S16). The imaged image, the concentration profiles, and the calculated HGB are displayed on the output part 27 (step S17).

In this manner, the hemoglobin concentration in the blood of the subject is thus measured.

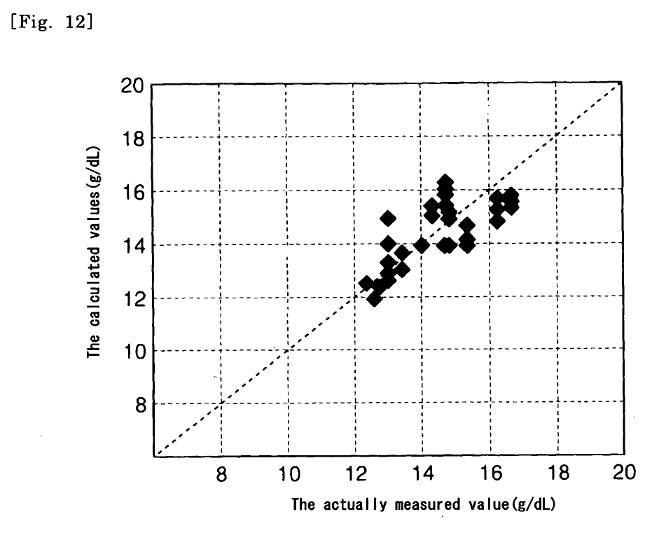

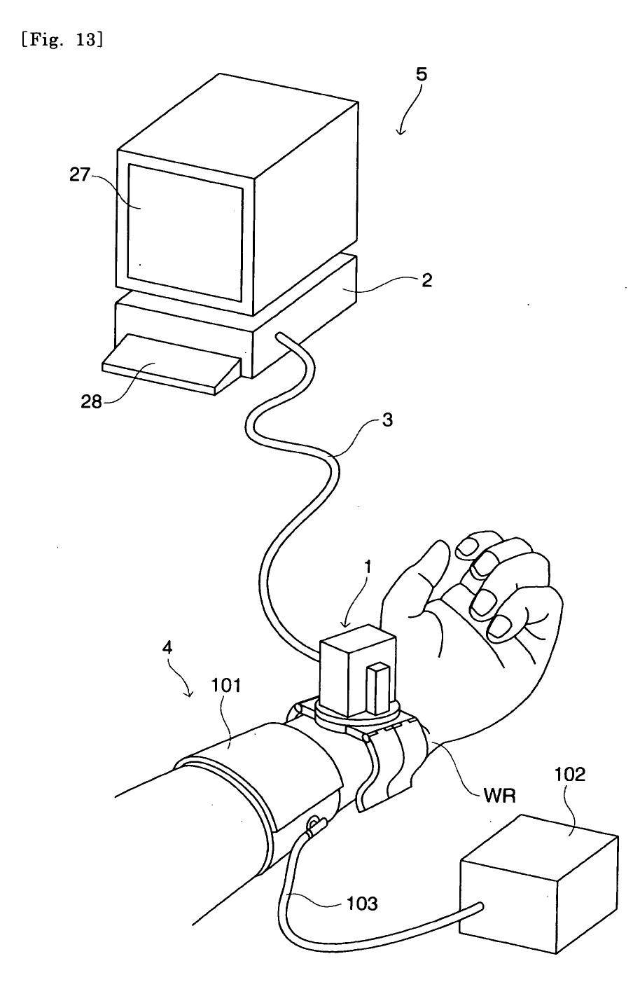

FIG. 12 is a graph plotting the actually measured values obtained from a blood counter and the calculated values of the measuring device of the present invention for the hemoglobin concentrations of plural subjects. It is found that the measuring device of the present invention can measure hemoglobin concentration with high accuracy.

The noninvasive biometric measuring device of this embodiment calculates the optimum target value of a press force applied to a living body to control the press force based on the optimum target value. The press force applied to the living body is properly maintained. It is possible to inhibit changing of the bloodstream of the measured region of the living body due to the measuring posture of the subject and the change of ambient temperature. Stable optical information can be obtained from the measured region to obtain a measured result with high accuracy.

In the above embodiment, the detecting part 1 and the analyzing part 2 are configured separately. The analyzing part 2 may be incorporated into the detecting part 1.

In the above embodiment, the analyzing part 2 analyzing optical information from the detecting part 1 and the controller 104 of the pressure controller 102 controlling a press force in the pressing band 101 are configured separately. The controller 104 may be incorporated into the analyzing part 2. In Second Embodiment, calculation of the optimum target value Tt of a pressure in the pressing band 101, control of a pressure applied to the pressing band 101 based on the optimum target value Tt (bloodstream control), and measurement of hemoglobin concentration can be continuously performed.

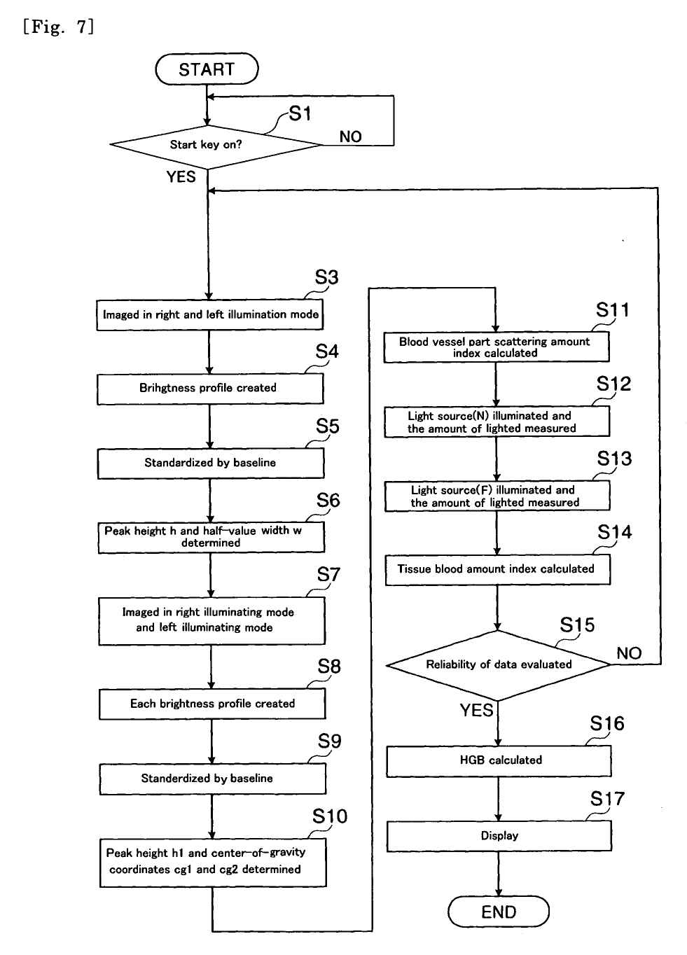

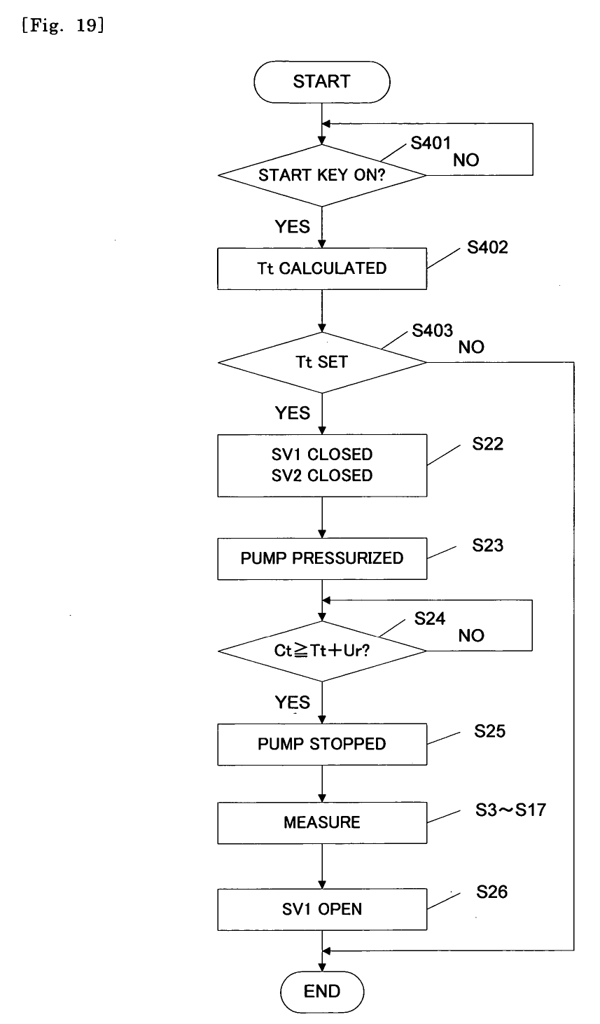

FIG. 19 is a flowchart of the noninvasive biometric measuring device according to Second Embodiment. According to Second Embodiment, the configurations of the biometric measuring device 5 and the living body pressing device 4 are the same as the above embodiment except that the controller 104 is incorporated into the analyzing part 2. In the control according to this embodiment, as shown in FIG. 19, the analyzing part 2 (see FIG. 13) judges whether the start switch, not shown, of the input part 2 is on or not (step S401). When the start key is on, the routine is advanced to step S402. In step S402, the optimum target value Tt of a pressure of the pressing band 101 is calculated to adjust the pressure of the pressing band 101 based on the optimum target value Tt. As the process of calculating the optimum target value Tt in step S402, a series of steps of calculating the optimum target value Tt shown in the flowcharts of FIGS. 16, 17, and 18 can be used. The valves SV1 and SV2 are then closed (step S22). The air pump is driven (step S23). The pressure Ct of the pressing band 101 is detected by the pressure sensor PD. When the pressure Ct is above the predetermined optimum target value Tt by the upper limit tolerance Ur, the air pump P is stopped (step S25). The routine is advanced to the hemoglobin level measuring step. The steps S3 to S17 shown in FIG. 7 are executed to open the valve SV1 for releasing air in the pressing band 101 into the atmosphere.

In the noninvasive biometric measuring device according to the second embodiment, the start switch alone is turned on to calculate the optimum target value of a press force applied to a living body, control the press force based on the optimum target value, and measure hemoglobin concentration. It is very simple.

According to the above embodiment, the living body is a mammal including a human, rabbit, dog, cat, rat, mouse, etc. The measured region is part of as is tissues of a living body, not tissues separated from the living body and includes such as the wrist, the palm of the hand, ankle, the sole of the foot, or the neck of a human.

As the light source part, a light source such as semiconductor laser (hereinafter, LD), LED, or halogen light source can be used and may be directly illuminated on part of a living body or may also be illuminated thereon via a fiber. The wavelength is preferably within the range of 400 to 950 nm.

The light receiving part can be configured of a light receiving device such as a photodiode or CCD. The light receiving part may include an optical system such as a lens.

The analyzing part can be configured of a microcomputer or personal computer.

1. A noninvasive biometric measuring device comprising:
an obtaining part obtaining biometric information from the measured region of a living body;
a pressing part for pressing the living body close to the measured region;
a pressure sensor detecting a press force of the pressing part;
a control part controlling the press force of the pressing part based on a detected value of the pressure sensor; and
an analyzing part analyzing the biometric information obtained by the obtaining part in such a state that the living body is pressed by the pressing part.

2. The noninvasive biometric measuring device according to claim 1, wherein biometric information is optical information.

3. The noninvasive biometric measuring device according to claim 2, wherein the obtaining part has a light source part illuminating a measured region including the blood vessel of a living body, and a light receiving part detecting optical information from the illuminated measured region.

4. The noninvasive biometric measuring device according to claim 1, wherein the control part sets a press force applied to a living body based on a detected value of the pressure sensor.

5. The noninvasive biometric measuring device according to claim 1, wherein pulsation is detected based on a detected value of the pressure sensor.

6. The noninvasive biometric measuring device according to claim 5, wherein the control part sets a level of press force between the largest press force and the smallest press force capable of detecting pulsation.

7. The noninvasive biometric measuring device according to claim 5, wherein the control part sets a press force applied to a living body based on detected pulsation.

8. The noninvasive biometric measuring device according to claim 4, wherein the control part controls a press force applied to a living body based on a set press force.

9. The noninvasive biometric measuring device according to claim 7, wherein the control part controls a press force applied to a living body based on a set press force.

10. The noninvasive biometric measuring device according to claim 2, wherein optical information is an image signal.

11. The noninvasive biometric measuring device according to claim 1, wherein the pressing part has a pressing member, an air pressure source applying an air pressure to the pressing member, and valves for decreasing the air pressure of the pressing member.

12. A noninvasive biometric measuring method comprising the steps of: pressing a pressed portion closer to a measured region of a living body; detecting a press force applied to the pressed portion; controlling said press force based on a detected value; obtaining biometric information from the measured region of the living body in such a state that the pressed portion is pressed; and analyzing the obtained biometric information.

13. The noninvasive biometric measuring method according to claim 12, wherein biometric information is optical information.

14. The noninvasive biometric measuring method according to claim 13, wherein the step of obtaining biometric information includes a step of illuminating a measured region including the blood vessel of a living body, and a step of detecting optical information in the illuminated measured region.

15. The noninvasive biometric measuring method according to claim 12, wherein the step of controlling the press force includes a step of setting a press force based on a detected value.

16. A living body pressing device comprising:
a pressing part adapted to adhere to a living body to press the living body;
a pressure sensor detecting a press force of the pressing part; and
a control part controlling the press force of the pressing part based on a detected value of the pressure sensor, wherein the control part calculates a press force applied to the living body based on the detected value to control the press force applied to the living body based on the press force.

17. The living body pressing device according to claim 16, wherein after calculating a press force applied to a living body based on the detected value, the press force applied to the living body is once released to press the living body again based on the calculated press force.

18. The living body pressing device according to claim 16, wherein pulsation is detected based on the detected value.

19. The living body pressing device according to claim 18, wherein the control part calculates a press force between the largest press force and the smallest press force capable of detecting pulsation.

20. The living body pressing device according to claim 16, wherein the pressing part has a pressing member, an air pressure source applying an air pressure to the cuff, and valves for decreasing the air pressure of the pressing member.
